(19) United States
(12) Patent Application Publication
(10) Pub. No.: US 2003/0073663 A1
(43) Pub. Date: Apr. 17, 2003

(54) BIOABSORBABLE MEDICAL DEVICES FROM OXIDIZED POLYSACCHARIDES

(76) Inventors: DAVID M WISEMAN, DALLAS, TX (US); LOWELL SAFERSTEIN, EDISON, NJ (US); STEPHEN WOLF, NESHANIC STATION, NJ (US)

Correspondence Address: AUDLEY A CIAMPORCERO JR, ONE JOHNSON AND JOHNSON, NEW BRUNSWICK, NJ, 08933

(21) Appl. No.: 08/881,984

(22) Filed: Jun. 25, 1997

(51) Int. Cl. A61K 31/715; A01N 43/04; A61K 31/665; A01N 57/00; A61K 31/727; A61F 2/00

(52) U.S. Cl. 514/54; 514/55; 514/56; 514/57; 514/58; 514/59; 514/60; 424/424

(57) ABSTRACT

Bioabsorbable medical devices are prepared by oxidizing derivatives of cellulose, including methyl cellulose, carboxymethylcellulose, and cellulose acetate. The resulting material is formed into films, sponges and, in the case of oxidized methyl cellulose, gels, due to its unique property of being water soluble. The resulting devices are particularly useful in limiting surgical adhesions, and for hemostasis. Other uses include wound dressings and as a replacement for more expensive bioabsorbable gels such as hyaluronic acid.

This application claims the benefit of U.S. Provisional Application No. 60/020,758 filed Jun. 28, 1996.

### BACKGROUND

1. Field of the Invention

The present invention relates generally to oxidized polysaccharides, especially derivatives of cellulose, and methods for their production and use.

2. State of the Prior Art

Surgeons face numerous problems during even routine surgery; one of the most frustrating is the formation of adhesions after a surgical procedure. An adhesion is a connection that occurs between two internal body surfaces that are not normally connected. Adhesions may occur for a number of reasons that are unrelated to surgery. However since adhesion formation may be regarded as being analogous to that of scar formation, it is not surprising that adhesions occur after surgery. It has been estimated that adhesion formation occurs in about 90% of all surgical procedures, and that about 10% of these cause post surgical problems (Ellis H The causes and prevention of intestinal adhesions. Br J Surg 69:241-243; 1982; Weibel M A, Majno G. Peritoneal adhesions and their relation to abdominal surgery. Am. J. Surg. 126:345-353,1973).

Depending on the anatomical site of adhesion formation different problems may arise. For example, adhesions involving the fallopian tubes may cause infertility. Adhesions involving the intestine may cause an intestinal obstruction. In the thorax following a cardiac procedure, adhesion formation may seriously complicate a redo sternotomy.

The typical approach to reducing adhesion formation is to limit trauma to the areas where adhesions form. However, even the most skilled surgeons induce sufficient trauma during many procedures to induce some degree of surgical adhesions. This is especially true during more invasive procedures such as open heart surgery. Also, some patients are more prone to form adhesions. At present, there are no approved drugs or devices which effectively reduce adhesion formation after cardiac surgery. However, a cloth of oxidized regenerated cellulose (INTERCEED available from Johnson & Johnson Medical, Inc.) has been approved by the United States Food and Drug Administration for reducing surgical adhesions in certain pelvic procedures and has been shown to have a general adhesion limiting effect in other surgical procedures.

Also, it has been suggested that certain water soluble gels made from hyaluronic acid or carboxymethylcellulose may be useful in limiting the formation of surgical adhesions. However, both these substances have certain disadvantages. Hyaluronic acid is somewhat difficult to produce. It is either purified from rooster combs, or it is produced by fermentation. Carboxymethylcellulose (CMC) is very inexpensive to produce, but does not degrade in the body. Like other cellulose derivatives such as methylcellulose and hydroxyethylcellulose, its metabolic fate is uncertain and may be sequestered by cells of the reticuloendothelial system (Hueper W C. Macromolecular substances as pathogenic agents. Arch. Pathol. 33:267-290,1942.; Hueper W C. Experimental studies in cardiovascular pathology. XI. Thesaurosis and atheromatosis produced in dogs by the repeated intravenous injections of solutions of sodium cellulose glycollate. Am. J. Pathol. 21:1021-1029,1945; Hueper W C. Experimental studies in cardiovascular pathology. XII. Atheromatosis in dogs following repeated intravenous injections of solutions of hydroxyethylcellulose. Arch. Pathol. 41:130-138,1946).

Carboxymethylcellulose (CMC) is a member of a class of cellulose derivatives that form water soluble gels. Cellulose itself may be rendered bioabsorbable by exposing it to oxidants. This was first discovered in 1936, by W. Kenyon of the Eastman Kodak Research Laboratories, who was carrying out fundamental research on the oxidation of cellulose. Kenyon found that a new type of product could be made by the use of nitrogen dioxide as an oxidizing agent. The material was soluble in alkali and in contrast to the usual friable materials resulting from other methods of oxidation of cellulose, this material maintained its original form and much of its original tensile strength. It was shown that the product was a copolymer of anhydroglucose and anhydroglucuronic acid. This oxidized cellulose material was developed into a bioabsorbable fabric hemostat by Parke Davis and Johnson & Johnson. A good discussion of the process can be found in the following article and patents, all of which are incorporated herein by reference: “Oxidation of Cellulose,” by Richard Kenyon, Industrial and Engineering Chemistry, vol 41 (1) 2-8, 1949; U.S. Pat. No. 2,232990 issued 1941; U.S. Pat. No. 2,298387 issued 1943; U.S. Pat. No. 3,364,2000 issued 1968 Ashton et al. U.S. Pat. No. 3,364,200, issued Jan. 16, 1968, and the Boardman et al. U.S. Pat. No. 5,180,398, issued Jan. 19, 1993 and its foreign equivalents, EP 0,492,990, Japanese Application No. 361083/91.

The oxidizing action of numerous oxidants on cellulose has been studied under widely varying conditions of temperature, pH, time of reaction and concentration. The major problem in studying oxidized celluloses is the difficulty of producing materials which are homogeneous in chemical and physical properties. Several of the oxidants employed are apparently not selective as to the particular hydroxyl groups of the anhydroglucose unit in the cellulose molecules which are attacked. Many methods of oxidation are topochemical. When the oxidation is mild, the products usually consist of an oxidized portion and an unchanged residue of unreacted or only slightly modified cellulose. More drastic oxidation produces a larger proportion of oxidized material accompanied by increased degradation. Physical degradation accompanying the oxidation breaks up the cellulose fibers and usually the material is friable and powders easily.

Although the earliest work on preparing oxidized cellulose with nitrogen dioxide goes back to the late 1930's - 1940's, we recently expanded the chemistry so that it is possible to prepare other bioabsorbable polymers from cellulose derivatives and other carbohydrates. We have recently discovered, that cellulose derivatives such as carboxymethylcellulose, water soluble cellulose monoacetate and methyl cellulose among others can also be oxidized with nitrogen dioxide to bioabsorbable materials. (A Czech Patent 118,765 dated Jun. 15, 1966 to Jozef Tamchyna and Frantisek Skoda discloses oxidizing starch with nitrogen tetroxide but there is no mention of bioabsorbability of the oxidized starch. We have also found that carbohydrates with the formula (C₆H₁₀O₅)ₙ such as guar, konjac, starch, and dextrin can also be oxidized with nitrogen dioxide to bioabsorbable polymers. We have also oxidized dextran, pustulan and cyclodextrin into bioabsorbable polymers. Alginates were also oxidized.

Methyl cellulose and carboxymethylcellulose (CMC) are two of the most widely used water soluble derivatives of cellulose and have applications in the food, cosmetic and pharmaceutical industries. They are made by the reaction of alkali cellulose with methyl chloride or chloroacetic acid respectively. The reaction conditions are usually chosen such that only partial substitution of the three hydroxyl groups on each cellulose monomer are substituted. Thus cellulose derivatives such as methyl cellulose and carboxymethylcellulose are partially substituted derivatives of cellulose. They dissolve in water to form thick, aqueous solutions, the viscosity of which depends upon the concentration of the polymer and its molecular weight. Methylcellulose has been commercialized by several companies, one of which is Dow Chemical Company who sells the product under the name METHOCEL A brand products. Carboxymethylcellulose is sold among others by Aqualon under the generic name sodium carboxymethylcellulose.

Neither methyl cellulose, carboxymethylcellulose nor cellulose acetate are bioabsorbable polymers. If a film, powder, sponge, solution or any device made of these polymers were placed within the body cavity, it would dissolve as a high molecular weight water soluble polymer. Although some excretion is possible, some of the polymer would eventually find its way into the walls of blood vessels, renal glomeruli and the cells of the reticuloendothelial system, possibly initiating damage thereto (Wiseman, D. M. Polymers for the Prevention of Surgical Adhesions. In: Polymer Site Specific Pharmacotherapy. Domb, A. (ed). John Wiley, Chichester, 1994 pp 385).

For a polymer to be bioabsorbable it must be broken down in some manner into low molecular weight fragments that can either be metabolized by the liver or excreted through the kidneys. For example, medical devices made from collagen and gelatin when placed within the body are enzymatically broken down into low molecular weight peptides and amino acid fragments which are metabolized by the liver into new proteins or are excreted by the kidneys. Polymers such as polyglycolide for absorbable sutures and polyanhydrides for controlled release of medicaments are sensitive to moisture and are broken down by body fluids to low molecular weight water soluble fragments that either pass out of the body in the urine or are metabolized by the liver. Oxidized cellulose is stable below pH 7, but as the pH approaches and exceeds that of body fluid, the polymer chain is broken down into water soluble low molecular weight oligosaccharides and sugar like moieties which are passed through the kidneys and eliminated into the urine. A good discussion of the pH sensitivity of oxidized cellulose is presented by Alexander Meller in “Holzforschung.” Vol. 14, pg. 78-89, 129-139 (1960).

### SUMMARY OF THE INVENTION

We have, discovered that not only can cellulose be converted to a bioabsorbable material through oxidation, but that other polysaccharides, and especially cellulose derivatives, can be oxidized with nitrogen dioxide into bioabsorbable polymers. The starting material and oxidizing process are relatively inexpensive when compared to naturally occurring bioabsorbable animal polymers such as hyaluronic acid and dextran. Further, they are useful in limiting surgical adhesions, and also for hemostasis, for the controlled release of drugs and as wound dressing materials among many medical uses.

A bioabsorbable material according to the present invention comprises an oxidized polysaccharide, preferably comprising a derivative of cellulose. Preferably, the derivative of cellulose is selected from the group consisting of: methyl cellulose, carboxymethylcellulose or cellulose acetate.

Preferably, the material is sterile and gamma irradiation is used as the preferred sterilization method. The material may be provided in the form of a film, gel, powder, fibrous mat or sponge among others.

In the case of oxidized methyl cellulose, it is both bioabsorbable and water soluble and may be mixed with a sufficient quantity of water or physiologically acceptable buffer to form a gel. The gel may be impregnated into a bioabsorbable substrate, such as a cloth formed of oxidized regenerated cellulose. The composite material may also be produced by coating a methyl cellulose solution onto a cloth of rayon or cellulose and drying to produce a film on the surface of the fabric or allowing the methyl cellulose solution to impregnate the interstices of the fabric. When dry the composite can be oxidized with nitrogen dioxide gas to produce a completely bioabsorbable fabric with a bioabsorbable gel forming polymer incorporated within its structure.

A process according to the invention for inhibiting adhesions comprises the step of applying a bioabsorbable material comprising an oxidized polysaccharide comprising a derivative of cellulose to a site on the body susceptible to adhesions. One preferred method comprises placing the material, most preferably a gel of oxidized methyl cellulose, into the thoracic cavity to inhibit cardiac adhesions. The gel may also be placed in the abdominal cavity, to prevent abdominal adhesions. The gel may also be applied to the body through a lumen in an endoscope.

### DETAILED DESCRIPTION

Of the cellulose derivatives, methyl cellulose is perhaps the most exciting in that it converts into a water soluble bioabsorbable polymer when reacted with nitrogen dioxide whereas the other oxidized cellulose derivatives we examined are water insoluble. The oxidized methyl cellulose polymer forms viscous aqueous gels at concentrations >3% which are stable below pH 7 but fall apart above pH 7 to thin watery solutions. The oxidation of methyl cellulose can be carried out in a number of ways, one of which involves exposing a slurry of methyl cellulose powder to a solution of nitrogen dioxide in an inert solvent such as carbon tetrachloride. The methyl cellulose slurry is exposed to the solution of nitrogen dioxide for 248, preferably 4-16 hours. At the end of the time period, the oxidized methyl cellulose powder is filtered away from the solvent and is washed in 90% isopropyl alcohol, 10% water, or 90% acetone, 10% water to remove the excess nitrogen dioxide gas. The oxidized methyl cellulose powder is dried with 100% acetone or 100% isopropyl alcohol to produce a white powder which then is water soluble. Solutions may be filtered to remove any insoluble material. Oxidized methyl cellulose solutions of concentrations between 0.5 and 5% can be cast onto glass with a casting blade and allowed to dry into films or the aqueous solution can be freeze dried into sponges. The oxidized polymer can also be precipitated from aqueous solutions by pouring the solution into acetone or isopropyl alcohol which are non-solvent for the polymer.

The chemistry of oxidation of methyl cellulose is speculated to be similar to that for oxidation of cellulose. A typical structure of methyl cellulose is shown below. The substitution of methyl ether groups on the hydroxyl groups of cellulose can occur on either the primary hydroxyl or the two secondary hydroxyl groups. The figure below shows an ideal structure with the methyl substitution only on the primary group but in fact METHOCEL A has a degree of substitution of 1.6-1.9, which means that more than one methyl group is substituted on each ring structure. The distribution of the methyl groups has been reported by Y. Tezuka, K. Imai, M. Oshima and T. Chiba, Macromolecules Vol. 20, pg. 2413-2418, 1987. When nitrogen dioxide comes in contact with the methyl cellulose polymer, oxidation of some of the secondary alcohol groups to ketones occurs along with oxidation of some of the unsubstituted primary alcohol groups to carboxylic acid. The oxidized methylcellulose polymer can be characterized by its carboxylic acid content which can range from about 3-8 percent.

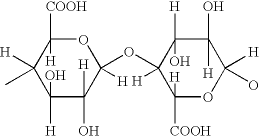

The discovery that a readily available low cost starting material as methyl cellulose can be converted to a bioabsorbable water soluble polymer has major implications in the area of bioabsorbable medical devices. For example recent teachings in the field of post-surgical adhesion prevention show that water soluble gels made from hyaluronic acid (Gel for Preventing Adhesion Between Body Tissues and Process for its Production, PCT/SE85/00282, Pharmacia Corp.; (Wiseman, D. M., Johns, D. B. Anatomical synergy between sodium hyaluronate (HA) and INTERCEED barrier in rabbits with two types of adhesions. (Fertil. Steril. Prog. Suppl. S25, 1993) or viscous solutions of carboxymethylcellulose (Viscoelastic fluid for use in spine and general surgery and other surgery and therapies and method of using same. Pennell P E, Blackmore J M, Allen M D. U.S. Pat. No. 5,156,839 Oct. 20, 1992; Assessment of carboxymethylcellulose and 32% dexran 70 for prevention of adhesions in a rabbit uterine horn model. Diamond, M P, DeCherney A H, Linksy, C B, Cunningham T, Constantine B. Int J Fertil 33;278-282, 1988). can be poured into the body cavity to coat tissue and organs with a viscous solution of these polymers. These viscous coatings prevent adjacent tissues from coming in contact with each other for a period of time of from one to ten days, sufficient to allow the tissue to heal and prevent adhesions from forming between the juxtaposed tissue. Viscous aqueous gels of oxidized methyl cellulose can function in the same way. The gel coats organs and tissue to prevent them from coming in contact with each other, then slowly falls apart as the body fluids buffer the solution to above pH 7.

In addition, solutions of oxidized methylcellulose may be used during surgery to minimize tissue damage to abrasion, desiccation and other incidental handling, as has been described for other materials (Goldberg E P and Yaacobi Y, Method for preventing surgical adhesions using a dilute solution of polymer, U.S. Pat. No. 5,080,893, Jan. 14 1992, incorporated herein by reference; Diamond, M P and the Sepracoat™ Adhesion Study Group: Precoating with Sepracoat™ (HAL-C™) reduces postoperative de novo adhesion formation in a multicenter randomized, placebo-controlled gynecologic clinical trial. J Soc Gynecol Invest 3;2 Suppl. 90A, 1996).

Sponges or films of oxidized methyl cellulose can be sterilized by gamma irradiation. When placed in the body they will slowly turn into a viscous gel and then gradually dissolve away as the polymer is degraded and solubilized into water soluble fragments. Sponges or films of oxidized methyl cellulose can be used as devices for the controlled release of medicaments by incorporating into the polymer solution a medicament and either casting a film, freeze drying the solution into a sponge, or using the viscous medicated solution as a gel. Oxidized methyl cellulose can also function as a replacement for synovial fluid for the lubrication of a joint, tendon sheath or bursa.

To prepare oxidized methyl cellulose films, methyl cellulose such as METHOCEL A polymer grades A15LV, A4C, A15C or A4M (available from Dow Chemical) with a degree of substitution of 1.65-1.92 is dissolved in water at 140° F. according to the manufacturer's instructions, at concentrations from 0.5%w/w to 15% w/w. The solution is cast onto a flat surface at nominal thicknesses from 5/1000″ to 200/1000″ and allowed to dry. The clear, pliable films are oxidized in either the gas phase with nitrogen dioxide gas or with a solution of nitrogen dioxide in an inert solvent such as carbon tetrachloride or Freon 113. In the preferred gas phase oxidation, the methyl cellulose films are placed in a resin kettle which is flushed with nitrogen gas to displace air. Chilled nitrogen dioxide of 1-3 times the weight of the films is placed in a small flask attached to the resin kettle via a side arm. The nitrogen dioxide gas is allowed to evaporate slowly into the resin kettle and envelop the films. The resin kettle is equipped with a cold condenser vented to the atmosphere which prevents pressure build up. The films of methyl cellulose are exposed to the nitrogen dioxide gas for a period of 248 hours, preferably 4-16 hours. At the end of this period the resin kettle is flushed with nitrogen to clear it of excess nitrogen dioxide gas and the films are removed. The oxidized films are washed in 90% isopropanol solution several times to remove adhering oxidant.

The resultant clear, pliable film material is found to dissolve in 0.5N NaOH to give a water thin solution, indicating that it is likely to be bioabsorbable. The oxidized film will also dissolve in water to produce a viscous, acidic, solution. The oxidized methyl cellulose is characterized by its carboxylic acid content which ranges from 3-8%. This oxidized film material was then sterilized by gamma irradiation (1.8 MRad) and implanted subcutaneously in rats. Macroscopic observation of the implant site 10 and 20 days post operative revealed no visible signs of the test material in any of the eight animals implanted with the oxidized methyl cellulose. Microscopic evaluation of the cellular response concluded that the test material was rapidly cleared from the subcutaneous implant. At 10 days postoperatively test material had provoked no unusual or unexpected cellular reaction. Sterilized films of oxidized methyl cellulose were also tested for their ability to prevent pericardial or abdominal adhesions.

The oxidized methyl cellulose film can also be impregnated onto a fabric for example a fabric composed of oxidized regenerated cellulose or an absorbable mesh made from lactide glycolide copolymer.

To prepare an oxidized methyl cellulose gel, methyl cellulose powder such as METHOCEL A4C, A15-LV or A4M available from Dow Chemical Company with a degree of substitution of 1.65-1.92 is spread evenly in a dish to a depth of 2-8 mm. The dish is placed inside a chamber which is flushed with nitrogen gas to displace the oxygen. Nitrogen tetroxide gas in amounts from one half to 3 times the weight of the methylcellulose powder is introduced into the chamber which is vented to a caustic trap to absorb the oxides of nitrogen. The exposure to nitrogen tetroxide may be carried out from two to forty eight hours. Following exposure the vessel is purged with nitrogen, the oxidized powder is washed with 70-90% isopropyl alcohol and air dried. Alternatively the methylcellulose powder can be suspended in an inert liquid such as Freon 113 in which the nitrogen tetroxide has been dissolved and this suspension stirred for 248 hours. The oxidized powder is separated from the inert liquid by filtering off the powder at the end of the reaction time period and washing the powder in 70-90% isopropyl alcohol. In the case of methylcellulose, the oxidized powder is soluble in water which may be cleared of some particulates by dissolving in water at a concentration of between 0.5 and 10% and filtering to remove insolubles. The resulting solution can be used as is or can be lyophilized to produce a sponge for testing.

Carboxymethylcellulose and cellulose mono-acetate are two other water soluble cellulose derivatives that have been converted into bioabsorbable polymers through the action of nitrogen dioxide. Cellulose acetate is water soluble when the degree of substitution of the acetate is below 1. This water soluble polymer from Celanese Corporation should be distinguished from the more common non-water soluble cellulose acetate used to make textile fabrics which has a degree of substitution of 2 or higher. Carboxymethylcellulose is produced by Aqualon in several grades with degrees of substitution between 0.38 and 1.45 and all grades are water soluble. When carboxymethylcellulose or cellulose acetate are oxidized with nitrogen dioxide, the resulting oxidized material is not water soluble but will dissolve in aqueous solutions with a pH>7 to yield thin, low viscosity solutions, indicating degradation and alkaline sensitivity of the oxidized polymer.

Carboxymethylcellulose and cellulose acetate can be made into films and sponges from dilute solutions of the polymer in water. Casting a dilute aqueous solution of carboxymethylcellulose or cellulose monoacetate on glass or plastic plates with a draw knife produces a film when dry. Freeze drying a dilute aqueous solution of between 0.5%-3% concentration will produce a sponge of these polymers. The films and sponges can be oxidized with nitrogen dioxide gas over a 16 hour period to produce the corresponding oxidized polymer which are now water insoluble and bioabsorbable. Films and sponges of either oxidized carboxymethylcellulose or oxidized cellulose acetate can be sterilized by gamma irradiation. These bioabsorbable devices can make good delivery systems for drugs, medicaments, barriers for adhesion prevention and absorbable hemostats to help arrest bleeding during surgery.

Other cellulose derivatives that may be oxidized include ethyl cellulose. The commercially available ethyl cellulose polymer from Hercules has a DS of 2.46 which is too high to allow sufficient primary and secondary hydroxyl groups on the cellulose backbone to become oxidized, and to be rendered bioabsorbable, as evidenced by dissolution in 0.5N sodium hydroxide. However if ethyl cellulose with a degree of substitution of 0.3 to 1.0 is oxidized with nitrogen tetroxide, it will be transformed into a bioabsorbable oxidized cellulose derivative.

### EXAMPLES

Examples 1-5 show methods of oxidizing cellulose derivatives by exposure to nitrogen dioxide in the gas phase. This oxidation may also be accomplished by exposure to nitrogen dioxide which is dissolved or carried in an appropriate solvent such as PF5060 as was taught by Boardman et al (U.S. Pat. No. 5,180,398).To ensure that the product does not dissolve during the washing phase, the amount of water in the washing solvent must be reduced to less than 50%, the remainder of the solvent being comprised of an alcohol such as isopropyl alcohol.

### Example #1

### Preparation of a Solution of Oxidized Methyl Cellulose

25 grams of METHOCEL A4M from Dow Chemical Co. was dissolved in 808 grams of distilled water to make a 3% solution. The solution was poured into trays and freeze dried to make sponges. The sponges were cut into small pieces and placed in a resin kettle. The kettle was flushed with nitrogen gas to displace the air for 5 minutes. 15 grams of chilled nitrogen dioxide was placed in a flask attached to the resin kettle by a side arm and the gas was allowed to diffuse into the sponges over a 24 hour period. At the end of this time period the sponges were removed from the resin kettle and allowed to degas. They were then washed 3 times, each with a one liter solution of 90% isopropyl alcohol and 10% water. The sponges were dried in 100% isopropyl alcohol. The dried oxidized sponges, 25 grams, were dissolved in 2475 grams of distilled water and filtered to remove any insolubles. The pH of this solution was 2.3. The clear solution was poured into trays and freeze dried. A 0.5 gram piece of dried sponge was dissolved in 10 ml of 0.5 N NaOH and titrated for percent carboxylic acid. This sample was found to have a 7 weight percent carboxylic acid content.

Ten grams of this sponge was dissolved in 115 grams of water to make an 8% solution. This viscous solution can be used to prevent abdominal and pelvic adhesions.

### Example #2

### Preparation of Oxidized Methyl Cellulose Films.

A 3% aqueous solution of METHOCEL A4M from Dow Chemical Company was poured onto a glass plate and drawn down with a draw knife set at 25 mils. The film was allowed to dry overnight and removed the next day from the glass plate to produce a 3 mil film. Three grams of the film were placed in a resin kettle which was flushed with nitrogen gas to displace the air. A small flask containing 2 grams of chilled nitrogen dioxide liquid was attached to the head of the resin kettle with a side arm adapter. The nitrogen dioxide was allowed to diffuse into the resin kettle to oxidized the film over a 16 hour period. At the end of this time the film was removed from the resin kettle and washed with a solution of 90% isopropyl alcohol and 10% water to remove any residual gas from the film. This film can be sterilized by gamma irradiation and used as a barrier for adhesion prevention. A 0.5g piece of the film dissolved in 10 ml of a 0. 5N sodium hydroxide solution to give a clear solution with low viscosity, indicating its likely bioabsorbability. Another 0.5g piece of film dissolved in 10 ml of water to give a solution with moderately high viscosity. This film can be sterilized by gamma irradiation and used as a barrier for adhesion prevention.

### Example #3

### Preparation of Oxidized Carboxymethylcellulose Sponges.

Ten grams of carboxymethylcellulose grade 7HF with a degree of substitution of 0.65-0.90 from Aqualon was dissolved in 490 grams of water to make a 2% solution. The solution was poured into 3×4 in. trays approximately one quarter inch deep. The solutions were frozen on the shelf of a freeze dryer and when fully frozen the vacuum pump was turned on to produce a vacuum of 100 millitore. The shelf temperature was adjusted to 15° C. and lyophilization was carried out for 20 hours. At the end of this time period the trays were removed from the freeze dryer and sponges of carboxymethylcellulose were obtained from each tray. These sponges were soft but still water soluble. The sponges, about 10 grams total, were placed in a resin kettle to which a side arm was connected to a small flask. The resin kettle was flushed with nitrogen gas for several minutes then 10 grams of chilled nitrogen dioxide was added to the small flask and slowly allowed to diffuse into the resin kettle containing the carboxymethylcellulose sponges. After 20 hours exposure to the nitrogen dioxide gas, the sponges were removed from the resin kettle and washed with 1 liter of a 50:50 solution of isopropyl alcohol and water. This washing was performed twice. The sponges were then placed in a tray under a source of running water and washed with fresh water for 5 minutes. The sponges were air dried after washing in 1 liter of 100% isopropyl alcohol or they could be freeze dried after washing in water. In freeze drying to make sponges, complete drying is desirable otherwise the sponge once formed may reabsorb moisture from the atmosphere.

The oxidized carboxymethylcellulose sponges are no longer water soluble. One gram however, dissolves in 10 ml of 0.5 N sodium hydroxide to give a clear solution with very low viscosity showing that the oxidized carboxymethylcellulose sponge degrades at the high pH of this sodium hydroxide solution. Titration of the sponge for carboxylic acid groups showed that there were more acid groups present after oxidation then before. The increase in carboxylic acid content due to ring oxidation alone is 6%. The sponges are soft, have fine pore size and will absorb 15 times their weight in water. These sponges can be sterilized with gamma irradiation and after sterilization sponges implanted subcutaneously in rats absorb within ten days with no visible tissue reaction. Oxidized carboxymethylcellulose sponges make good hemostats. When tested on a swine splenic incision model, these sponges will stop bleeding in an average of 2.5 minutes compared to oxidized cellulose fabric (SURGICEL) which stops bleeding in 6.5 minutes. Sponges of oxidized carboxymethylcellulose can also be used to fill tumor beds, to cover wounds, as diaphragms for contraceptive control and as drug delivery systems. This method is preferred for the production of sponges of oxidized methylcellulose.

### Example #4

### Preparing Films of Oxidized Carboxymethylcellulose

Solutions of carboxymethylcellulose can be cast on glass plate with a draw blade and allowed to dry to give films which also can be oxidized by exposure to nitrogen dioxide gas. Films of oxidized carboxymethylcellulose can be used as absorbable barriers to prevent adhesions.

A 3% aqueous solution of carboxymethylcellulose grade 7MF with a DS of 0.90 from Aqualon was prepared and cast on a glass plate with a draw blade set at 30 mils openings. The film was allowed to air dry overnight. The dry films (3 grams) were placed in a resin kettle which was flushed with nitrogen for 5 minutes to remove the air. A side arm was attached to the resin kettle which connected to a small flask containing 3 grams. of chilled nitrogen dioxide. The liquid nitrogen dioxide was allowed to vaporize and diffuse into the flask containing the films of carboxymethylcellulose. The films were exposed to the gas for a period of 20 hours. At this point they were removed from the resin kettle and washed with water until the pH of the washings were above 3. The films were dried with isopropyl alcohol or acetone. Some were plasticized with 100/% glycerol which imparted flexibility to the dry films. The films are water insoluble but dissolve in sodium hydroxide. When titrated for carboxylic acid content they are found to have a 26 weight percent acid content. The increase in carboxyl content from ring oxidation is 7%. The films also absorb within ten days when implanted subcutaneously in rats. These absorbable films make good barriers for the prevention of adhesions.

### Example #5

### Preparation of Oxidized Cellulose Acetate Devices

A 2% aqueous solution of water soluble cellulose monoacetate available from Celanese Corporation was prepared and poured into trays. The solutions were freeze dried in a lyophilizer for 24 hours. White, soft, sponges were prepared by this procedure. The sponges (3 grams) were placed in a resin kettle to which a side arm was connected to a flask. The resin kettle was flushed with nitrogen for 5 minutes. The flask was then filled with 3 grams of chilled nitrogen dioxide and the liquid was allowed to vaporize and diffuse into the cellulose acetate sponges. After 20 hours exposure to the gas, the sponges were removed and washed with water until the pH of the wash water was above 3.0. The sponges were dried by washing two times with 1 liter of isopropyl alcohol. The sponges are white, soft, and non water soluble. They will dissolve in sodium hydroxide and titration of the sponge for carboxylic acid groups showed a 13.3% carboxylic acid content. The sponges were tested for hemostatic efficacy and were found to stop bleeding on a swine splenic incision model in 3 minutes when averaged over 6 tests compared to oxidized cellulose fabric (SURGICEL) which stopped bleeding in 6 minutes in this same test.

Films of cellulose acetate can also be prepared by casting an aqueous solution of the polymer onto a glass plate and allowing the solution to dry. Gaseous oxidation of the cellulose acetate films will produce oxidized cellulose acetate films which are no longer water soluble. Oxidized cellulose acetate is a bioabsorbable material and can function as a hemostat, wound dressing, adhesion barrier, controlled release device and in other medical functions wherever a bioabsorbable device is needed.

### Example #6

### Preparation of a Composite Oxidized Methylcellulose and Oxidized Cellulose Fabric

A dilute aqueous solution of 1.0% concentration of METHOCEL Al 5LV from Dow Chemical Company with a degree of substitution of 1.65 was prepared by dispersing 1.0 grams of methylcellulose powder in 30 grams of water heated to 95° C. Mixing was continued until all the particles were thoroughly wetted. To this dispersion was added 69 grams of cold water and stirring was continued until all the powder had dissolved and viscosity increased.

A five gram piece of a knitted rayon fabric was dipped into the METHOCEL A15-LV solution for 20 seconds then removed and allowed to dry. The dry impregnated cloth weighed 5-25 grams indicating a 5% pick up of methyl cellulose.

The dry methyl cellulose impregnated fabric was placed in a resin kettle which was flushed with nitrogen gas to displace air and then exposed to 15 grams of nitrogen dioxide gas for a period of 16 hours. At the end of this period the resin kettle was purged of the excess nitrogen dioxide by a nitrogen purge which displaced the oxidant to a caustic trap to neutralize the nitrogen dioxide. The fabric was removed and washed in 300 ml of isopropyl alcohol several times to remove adhering oxidant. The fabric was then allowed to air dry. Titration of a sample of the fabric for carboxylic acid content indicated an 18% carboxylic acid content and complete solubility in 0.5N sodium hydroxide. The oxidized composite of oxidized cellulose fabric impregnated with oxidized methylcellulose can be used to prevent postsurgical adhesions.

### Example #7

### Preparation of a Composite Oxidized Carboxymethylcellulose and Oxidized Cellulose Fabric

A 2.5% aqueous solution of carboxymethylcellulose, grade 9M8 from Aqualon with a degree of substitution of 0.8-0.95 was cast onto a woven rayon fabric and drawn down with a draw blade and allowed to dry on the surface of the woven fabric so that many of the pores of the fabric were bridged by a thin film of carboxymethylcellulose. The dry woven rayon fabric with its thin film of carboxymethylcellulose on the surface (weighing a total Of 10 grams) was placed in a resin kettle equipped with a magnetic stir bar and a condenser vented to a caustic bath to prevent pressure build up. A side arm was attached to the head of the resin kettle which led to a small flask in which was placed 15 grams of cold nitrogen dioxide. The nitrogen dioxide was allowed to slowly evaporate into the resin kettle and envelop the composite of woven rayon and CMC film. After 20 hours of exposure the excess gas was vented out with nitrogen gas and the fabric removed and washed in 500 ml of isopropyl alcohol several times The fabric was allowed to air dry and a small sample was titrated for acid content and found content and found to have 20% carboxylic acid content. The oxidized sample was completely soluble in 0.5N sodium hydroxide. The oxidized CMC film on the surface of the oxidized woven rayon fabric was difficult to delaminate. When implanted subcutaneously in rats, the material was absorbed within 28 days with minimal tissue reaction. The composite can be used to prevent post surgical adhesions.

### Example #8

### Preparation of Powders of Oxidized methyl or Oxidized Carboxymethylcellulose

A slurry of carboxymethylcellulose or methyl cellulose powder is made in an inert solvent such as carbon tetrachloride. The slurry is exposed to a solution of nitrogen dioxide in the inert solvent for 248 preferably 4-16 hours. At the end of the time period, the oxidized methyl or carboxymethylcellulose powder is filtered away from the solvent and is washed in 90% isopropyl alcohol, 10% water, or 900% acetone, 100% water to remove the excess nitrogen dioxide gas. The oxidized methyl or carboxymethylcellulose powder is dried with 100% acetone or 100% isopropyl alcohol to produce a white powder. The oxidized methylcellulose powder will form an aqueous solution which may be filtered to remove any insoluble material. Oxidized methyl cellulose solutions of concentration between 0.5 and 5% can be cast onto glass with a casting blade and allowed to dry into films or the aqueous solution can be freeze dried into sponges. The oxidized methylcellulose polymer can also be precipitated from aqueous solutions by pouring the solution into acetone or isopropyl alcohol which are non-solvents for the polymer. Bioabsorbable glove lubricants, instrument lubricants and mold release agents may be made by this method.

### Experimental Methods

All animals are assigned blindly and randomly to a treatment group which is revealed to the surgeon only upon completion of the abrasion. All evaluations are made on a blinded basis.

### The Rabbit Pericardial Adhesion Model

Under anesthesia the thorax of white New Zealand rabbits is entered via a midline sternal incision. The pericardium is similarly opened and the anterior surface of the heart abraded forty times using a piece of gauze wrapped around the forefinger. If an absorbable fabric or barrier such as the present invention is to be used, then an elliptical piece with axes 2″×3″ approximately, is placed over the anterior surface, of the heart and may be sutured to the pericardium if desired. The thorax is closed in layers.

Twenty three to thirty days later the animals are sacrificed and the adhesions between the anterior surface of the heart and the underside of the sternum evaluated for adhesions. The percentage adhesion involvement of a strip 1 cm wide and extending from the apex to the base of the anterior cardiac surface is estimated. This strip represents the surface of the heart in intimate contact with the sternum and where adhesions are most likely to occur and cause a problem for the surgeon attempting a reentry to the thorax.

### The Rabbit Uterine Horn Model

The rabbit uterine horn adhesion model was performed as described by Linsky et al. Linsky C B, Diamond M P, Cunningham T, Constantine B, DeCherney A K, dizerega G. Adhesion reduction in the rabbit uterine horn model using an absorbable barrier—TC7. J. Reprod. Med. 32:17-20,1987). Under appropriate anesthesia and aseptic technique and using female White New Zealand rabbits (2-2.6 kg) the abdomen was entered via a ventral midline incision. Using a number 10 scalpel blade, 5 cm lengths on both sides of each uterine horn, starting approximately 1 cm from the uterine bifurcation, were scraped 20 times. Haemostasis was achieved by tamponade. The wound was closed in layers. Two weeks later the animals are euthanised and adhesions evaluated “blindly” according to the following standard system.

The extent of adhesions was quantified by measuring the length of uterine horn which had adhesions. The following grades were assigned:

0=No adhesions

1=25% of traumatized area with adhesions

2=50% of traumatized area with adhesions

3=100% of traumatized area, i.e. 5 cm length involved with adhesions.

The tenacity of adhesions was graded as follows:

0.0=No resistance to separation

0.5=Blunt dissection required for separation

1.0=Sharp dissection required for separation

The tenacity and extent scores were added to give a possible maximum score of 4, representing both extent and severity of adhesions.

### Experimental Results

### Cardiac Adhesion Model

A film of methyl cellulose (Grade A15C from Dow Chemical) was oxidized in the gas phase by exposure to nitrogen tetroxide gas. After washing and irradiation it was tested in the rabbit cardiac adhesion model by wetting in saline and then pressing onto a sheet of INTERCEED ® (TC7) Absorbable Adhesion Barrier. An elliptical piece of this composite (2×1″ approx) was placed over the anterior surface of the heart. Four weeks later the adhesions were evaluated and a significant reduction in adhesion formation was noted.

| | Animal No. | Percent adhesions |
|---|---|---|
| Control (no treatment) | | |
| | 217.03 | 100 |
| | 218.33 | 100 |
| | 218.49 | 80 |
| | 218.04 | 90 |
| | Mean | 92.5 |
| | SD | 9.57 |
| | N | 4 |
| Oxidized methyl cellulose | | |
| on INTERCEED ® Barrier | | |
| | 217.04 | 50 |
| | 217.02 | 100 |
| | 219.53 | 80 |
| | 218.32 | 15 |
| | 218.03 | 10 |
| | Mean | 51 |
| | SD | 39.4 |
| | N | 5 |

Using Student's t-test, there was a statistically significant reduction in adhesion formation when the oxidized methyl cellulose/INTERCEED Barrier composite was used (P<0.05). Historical results from a number of experiments gives an average adhesion score of approximately 80%.

### Oxidized Methyl Cellulose (OMC) Gel in the Rabbit Uterine Horn Model

Methyl cellulose powder (METHOCEL Grade A4M from Dow Chemical) was oxidized in the gas phase and sterilized by irradiation (1.8 MRad) on dry ice. To 7.5 g of this sterile material were added 115 ml of sterile water and 3 ml of glycine sodium (5% w/v) as a buffer. Unlike the non-oxidized material which dissolves slowly, the oxidized methylcellulose powder dissolved rapidly and easily with shaking for one minute to form a viscous solution.

For comparison a 2% w/w solution of sodium carboxymethylcellulose (CMC) (Hercules, 7H4F) was made using a blender. This is a non-degradable material that forms a solution only after vigorous agitation. carboxymethylcellulose was used as a positive control as it has been shown previously to reduce adhesion formation (Viscoelastic fluid for use in spine and general surgery and other surgery and therapies and method of using same. Pennell P E, Blackmore J M, Allen M D. U.S. Pat. No. 5,156,839 Oct. 20, 1992; Diamond, M. P., DeCherney, A H., Linsky, C. B., Cunningham, T., Constantine, B., Assessment of carboxymethylcellulose and 32% dextran 70 for prevention of adhesions in a rabbit uterine horn model. Int. J. Fert. 33;278-282, 1988).

The carboxymethylcellulose and oxidized methylcellulose solutions were of comparable viscosity. The standard rabbit uterine horn model was performed. Just prior to closure, 20 ml of either oxidized methylcellulose or carboxymethylcellulose the solutions were instilled into the abdominal cavity. Two weeks later adhesions were evaluated blindly.

| | % with no adhesions | N | P |
|---|---|---|---|
| Control | 17 | 12 | |
| Carboxymethylcellulose | 42 | 12 | 0.0331 |
| (not degradable) | | | |
| Oxidized | 50 | 20 | 0.0055 |
| methyl cellulose | | | |
| (degradable) | | | |

N = number of horns evaluated
P = one tail probability using Chi-squared test, comparing treatment with control

N=number of horns evaluated

P=one tail probability using Chi-squared test, comparing treatment with control

This result shows that a degradable gel of oxidized methylcellulose is able to reduce adhesions to a degree comparable to the effect found by carboxymethylcellulose.

The present invention provides many advantages over the prior art. For instance, prior teachings regarding cellulose derivatives appear limited to those which are not degradable by hydrolysis but are probably eliminated from the body intact, or are sequestered by the endothelial system. The materials of the present invention provide similar chemical or physical properties but may degrade into small excretable fragments by simple hydrolysis.

In the form of a film or sheet, these materials may be useful for the prevention of adhesions or as hemostats. The materials may also be formed into sponges, primarily for use as hemostats. However, a sponge of oxidized methylcellulose placed into a specific location within the body, would quickly dissolve into a protective gel. These sponges can also be used to absorb fluid from exuding wounds since they absorb at least times their weight of saline. Films or sponges may be used as a primary wound dressing to protect the wound and to provide a moist environment for healing.

All of these materials possess acidic groups which may render them bactericidal. This is a particular advantage in the reduction of infection. The acidic sites may also make these materials suitable for binding with certain drugs, providing a matrix for their controlled released delivery. For example, growth factors may bind to the acidic groups and are subsequently released into a body cavity by ion exchange or as the polymer degrades.

The viscoelastic nature of these materials makes them useful for a number of applications in addition to adhesion prevention or drug delivery by acidic binding. The viscous nature of solutions or sheets of these materials alone may be useful for drug delivery. The materials could also be useful as a degradable surgical lubricant for instruments and gloves. The use of these degradable materials as a mold release agent for medical devices or as a powder for glove lubrication is a distinct advantage over the use of talc or starch which some glove users prefer to avoid. Powders of oxidized methyl- or carboxymethylcellulose may be prepared by the slurry method described in example 8

The materials provide characteristics similar to other materials which are either more expensive or difficult to characterize because they are derived from natural or fermentation sources. Such materials include hyaluronic acid, heparin, chondroitin sulfate and dextran. Thus the relatively inexpensive and easy-to-standardize materials of the present invention may be used as blood expanders, and viscoelastic aids in ocular and orthopedic surgery and tissue augmentation materials. The materials produced may be sterilized by irradiation.

Cross-linking and derivitisation methods known in the art may be used to refine the characteristics of the materials described by this invention, or to bind, covalently or otherwise, certain drugs to the materials. The use of crosslinking agents such as dimethylolurea (bis(N-(hydroxymethyl urea)), divinyl sulfone etc. with the oxidized cellulose derivatives will slow down their bioabsorbtion and will allow them to last in the body longer. In some cases this attribute may enhance their performance. A lightly crosslinked gel of oxidized methyl cellulose will have higher gel strength and be somewhat firmer than non-crosslinked gels.

Oxidized materials can be used as delivery vehicles for drugs, and further may have pharmacological effects on their own. In the following experiment the effects of oxidized regenerated cellulose alone and as impregnated with heparin were examined with respect to the control of restenosis after trauma to a blood vessel. Similar results would be expected for other oxidized cellulose materials.

The endothelium is stripped from a length of the common carotid artery in anesthetized rats. The portion of the blood vessel which was injured is then wrapped with a piece of INTERCEED oxidized regenerated cellulose or with INTERCEED which is impregnated in situ with 400 U of heparin sodium injection USP. A third group has no treatment post-injury. A fourth group has 400 U heparin injected around the injury site. Each group contained 5 animals. Measurements are made of areas of lumen, intima and media on cross sections of arteries recovered 20 days after surgery.

The results follow, in which the numeric values represent the mean cross sectional area in square micrometers:

| | Media | Intima | Lumen | Bleeding |
|---|---|---|---|---|
| Control | 194,517 | 268,672 | 380,241 | None |
| INTERCEED alone | 225,146 | 161,011 | 549,936 | None |
| INTERCEED and heparin | 218,260 | 107,529 | 630,026 | Mild |
| | | | | hematoma |
| Heparin alone | | | | Death from |
| | | | | bleeding |

Two animals which were treated only with heparin died of hemorrhage. Thus, this control group was not completed. However, only mild haematomas in some of the animals treated with the same dose of heparin plus INTERCEED indicates that heparin was sequestered at the implantation site in the presence of INTERCEED.

Disrupted endothelium associated with vascular surgery can produce thrombosis and restenosis at the site. This is a major complication of any vascular surgery. Systemic delivery of drugs such as heparin to treat these conditions is not satisfactory due to the high dosage levels required. Thus, local delivery is the preferred route. Our results show that a matrix of oxidized regenerated cellulose impregnated with heparin provides controlled local delivery of heparin and a significant reduction in the damaging effects of trauma to the artery.

Significantly, the oxidized regenerated cellulose alone produced substantial results over the control group, indicating that oxidized regenerated cellulose has pharmacological activity on its own.

While the invention has been particularly described in connection with specific embodiments thereof, it is to be understood that this is by way of illustration and not of limitation, and that the scope of the appended claims should be construed as broadly as the prior art will permit.

The oxidation of cellulose derivatives to produce bioabsorbable polymers is not limited to the examples given here, but can be expanded to other derivatives such as those containing alipahtic groups attached to the cellulose ring.

What is claimed is:

1. A bioabsorbable material comprising oxidized methyl cellulose with a degree of substitution before oxidation of 0.50 to 1.92 and wherein the oxidized methyl cellulose is bioabsorbable and water soluble with a carboxylic acid content from ring oxidation between 3 and 10%.

2. A material according to claim 1 further comprising a sufficient quantity of water or physiologically acceptable buffer to form a gel.

3. A material according to claim 1 formed by oxidizing methyl cellulose in the presence of an oxidizer selected from the group consisting of nitrogen dioxide or nitrogen tetroxide.

4. A material according to claim 1 wherein the carboxylic acid content of the oxidized methyl cellulose is about from 3-10 percent.

5. A material according to claim 1 which is sterile.

6. A material according to claim 1 further comprising a sufficient quantity of water to form a gel and wherein the gel of the water and oxidized methyl cellulose is impregnated into a cloth formed of a second bioabsorbable material.

7. A material according to claim 6 wherein the second bioabsorbable material is oxidized cellulose.

8. A material comprising an oxidized polysaccharide.

9. A material according to claim 8 wherein the polysaccharide is selected from the group consisting of; ethyl cellulose, methyl cellulose, carboxymethylcellulose or cellulose acetate.

10. A material according to claim 9 wherein the polysaccharide comprises carboxymethylcellulose with a degree of substitution between 0.38 and 1.45

11. A material according to claim 10 wherein the carboxylic acid content due to the oxidation of the primary alcohol of the cellulose structure is 3 to 12% by weight.

12. A material according to claim 8 in the form of film wherein the oxidized film material is plasticized with polyhydroxy alcohols such as glycerol or propylene glycel to impart flexibility.

13. A material according to claim 8 wherein the polysaccharide comprises methyl cellulose.

14. A material according to claim 8 wherein the polysaccharide comprises cellulose acetate.

15. A material according to claim 8 wherein the polysaccharide is selected from the group consisting of: starch, alginates, guar, konjac, dextrin, dextran, pustulan and cyclodextrin.

16. A material according to claim 8 which is sterile.

17. A material according to claim 8 in the form of a film.

18. A material according to claim 8 in the form of a gel.

19. A material according to claim 9 in the form of a freeze dried sponge.

20. A process for inhibiting adhesions comprising the steps of:
preparing a bioabsorbable material by oxidizing a polysaccharide comprising a cellulose derivative; and
applying the oxidized bioabsorbable material to a site on the body susceptible to adhesions.

21. A process according to claim 20 and further comprising the step of sterilizing the bioabsorbable material.

22. A process according to claim 20 and wherein the oxidizing step comprises placing the cellulose derivative into contact with an oxidizer selected from the group of nitrogen dioxide and nitrogen tetroxide.

23. A process according to claim 20 wherein the cellulose derivative is selected from the group consisting of: ethylcellulose, methyl cellulose, carboxymethylcellulose or cellulose acetate.

24. A process according to claim 20 wherein the carboxylic acid content due to the oxidation of the primary alcohol of the cellulose derivative is 3 to 12% by weight.

25. A process according to claim 23 wherein the cellulose derivative comprises carboxymethylcellulose with a degree of substitution between 0.38 and 1.45.

26. A process according to claim 23 wherein the cellulose derivative comprises ethyl cellulose with a degree of substitution between 0.3 and 1.0.

27. A process according to claim 23 wherein the cellulose derivative comprises cellulose acetate with a degree of substitution between 0.3 and 1.0.

28. A process according to claim 23 wherein the cellulose derivative comprises methyl cellulose with a degree of substitution between 0.5 and 1.92.

29. A process according to claim 28 and further comprising the step of including a sufficient quantity of water or a physiologically acceptable buffer to form a gel of the bioabsorbable material.

30. A process according to claim 27 and further comprising the step of impregnating the gel onto a bioabsorbable substrate.

31. A process according to claim 30 wherein the cellulose derivative is first impregnated onto a cellulosic fabric precursor of the substrate and then they are oxidized together.

32. A process according to claim 30 wherein the substrate comprises a fabric composed of a bioabsorbable material.

33. A process according to claim 32 wherein the bioabsorbable material of the fabric comprises: oxidized cellulose, poly lactide-co-glycolide, polydioxanone, polycaprolactone, polyanhydride, ploylactide, polyglycolide, gelatin, collagen, elastin, polyphospazene, hyaluronic acid, polyorthoester, or a combination thereof.

34. A process according to claim 32 wherein the substrate comprises oxidized cellulose.

35. A process according to claim 34 and further comprising the step of placing the gel and substrate into the thoracic cavity to inhibit cardiac adhesions.

36. A process according to claim 29 and further comprising the step of placing the gel or article into the pelvic, peritoneal, cranial thoracic or other body cavity, or around tendons, ligaments or the eyes to inhibit adhesions.

37. A process according to claim 29 and further comprising the step of applying the gel to the site on the body through a lumen in an endoscope.

38. A process according to claim 28 and wherein the oxidizing step comprises placing the cellulose derivative in contact with an oxidizer selected from the group of nitrogen dioxide and nitrogen tetroxide.

39. A process according to claim 38 wherein the methyl cellulose is in the form of a sponge prior to the step of oxidation.

40. A process according to claim 39 and after the step of oxidation, further comprising the step of washing the methyl cellulose in a solvent for the oxidizing agent but a non-solvent for the oxidized methyl cellulose.

41. A process according to claim 28 wherein the solvent is selected from the group comprising: isopropyl alcohol and water.

42. A process according to claim 28 further comprising the step of dissolving the oxidized methyl cellulose in water to form a solution containing 1-10% oxidized methylcellulose.

43. A process according to claim 28 and further comprising the steps of: dissolving the oxidized methyl cellulose in water to form a solution; casting the solution onto a surface; and drying the cast solution to form a film.

44. A process according to claim 28 and further comprising the steps of: dissolving the oxidized methyl cellulose in water to form a solution; and freeze drying solution to form a sponge.

45. A process for inhibiting adhesions comprising the steps of:
preparing a bioabsorbable material by impregnating a cellulose derivative on the surface of a cellulose fabric and oxidizing this composite material to produce a bioabsorbable material; and
applying the bioabsorbable material to a site on the body susceptible to adhesions.
